(19) United States
(12) Patent Application Publication
(10) Pub. No.: US 2007/0244418 A1
(43) Pub. Date: Oct. 18, 2007

(54) Vibrator sex toy conversion

(76) Inventor: Stuart Harkness, Moffat (GB)

Correspondence Address: BELASCO, JACOBS & TOWNSLEY LLP;HOWARD HUGHES CENTER, 6100 CENTER DRIVE, SUITE 630, LOS ANGELES, CA, 90045, US

(21) Appl. No.: 11/403,581

(22) Filed: Apr. 13, 2006

(51) Int. Cl. A61H 1/00; A61F 5/00

(52) U.S. Cl. 601/72; 601/70; 601/80; 600/38

(57) ABSTRACT

A vibrator sex toy conversion includes a vibrating device having a power source, a motion generator, a handle and an actuating member removably attached to the vibrating device. In one embodiment, the actuating member is a replacement for the removable handle member of an electric toothbrush. The actuating member has a proximal end and a distal end. The proximal end is adapted to fit the handle member and the distal end is sized and shaped for tactile stimulation. In a second embodiment, the actuating member is a cover adapted to fit removably over a portion of a removable toothbrush member. The actuating member is formed of resilient material, may contain a plurality of ridges and grooves, and may include a plurality resilient protruding elements. In appearance, the conversion appears to be an ordinary electric toothbrush.

### FIELD OF INVENTION

The invention pertains to devices for sexual stimulation. More particularly, the invention relates to conversion systems for use with conventional electric toothbrushes.

### BACKGROUND OF THE INVENTION

As airline and other travel security searches become increasingly invasive, those who enjoy the use of various types of sexual stimulation devices have become subject to unwanted scrutiny and potential embarrassment as their luggage is examining when they carry such devices with them. The present invention is designed to ameliorate this problem by providing a series of innocuous appearing conversion devices for use with standard and readily available electric toothbrushes.

Various sex toys and sexual stimulation devices have been developed, some based on other existing devices. U.S. Pat. No. 5,470,303, issued to Leonard et al. discloses a novel device that includes a housing containing a drive and carrying a soft, flexible, resilient, tongue-shaped head on one end thereof, the head containing an arcuate shaft with a straight inner end connected to the drive, whereby the path of travel of the shaft during rotation defines an ellipsoid with a cone on the outer free end thereof.

U.S. Pat. No. 6,902,525, issued to Jewell describes a device designed to take rotary power from an external power source such as a cordless electric screwdriver and transmit that energy into both rotary and reciprocating linear activation of sexual devices used by both males and females for the purpose of masturbation and auto-stimulation is disclosed. The combination of rotational stimulation with reciprocating linear stimulation is apparently remarkably satisfying despite the absence of such stimulation during normal person-on-person sexual contact. The device is simple, light weight, small, powerful, modestly priced, handheld or hands-free, user serviceable, hygienic, gender neutral, sexual orientation neutral, convenient, rugged, multi-motion, safe, reliable, quiet, fully submersible, positionally versatile and sexually satisfying.

U.S. Pat. No. 6,895,625, issued to Lev et al. discloses a motorized electric toothbrush that includes a removable brush head and a switch that allows the toothbrush to be used in an “automatic” mode. The “automatic” mode provides for intermittent motorized operation, such that when the brush head contacts the operator's teeth, a switch is actuated and the motor is engaged. The brush head is easily removed for replacement when the bristles become worn or to allow use of the toothbrush by multiple users.

U.S. Pat. No. 6,833,639, issued to Lau et al. is directed to an electric actuator for providing an angular reciprocating drive, comprising a casing, a stator housed in the casing, and an armature extending co-axially through the stator. The stator has an annular arrangement of poles and a coil for energization by an alternating current to magnetise the poles in opposite polarities as between adjacent poles. The armature has a shaft supported by the casing for angular movement and a plurality of permanent magnets mounted fast on and around the shaft. The magnets have proximal sides as between adjacent magnets of opposite polarities, wherein upon repeated reversals of said alternating current the magnets interact magnetically with the poles to cause the shaft to oscillate thereby providing said drive.

U.S. Pat. No. 6,833,639, issued to Lau et al. discloses an electric actuator. According to the invention, there is provided an electric actuator for providing an angular reciprocating drive. The invention also provides an electric hand tool incorporating said electric actuator, including a body housing the actuator and an implement connectable to an output end of the shaft for driving thereby to reciprocate for operation. For example the electric hand tool is an electric toothbrush, in that the body comprises a handle and the implement comprises a brush head.

U.S. Pat. No. 3,991,751, issued to O'Rourke is directed to a tubular housing that is dimensioned to be held in a hand and has an open neck end. The housing has a predetermined diameter throughout its length and a diameter smaller than the predetermined diameter at the neck end thereof and is rounded down at its neck end from the predetermined diameter to the smaller diameter. A head is removably affixed to the housing at the neck end thereof and has the predetermined diameter at an intermediate part thereof. The head has the smaller diameter at the end affixed to the housing and is rounded down at its affixed end from the predetermined diameter to the smaller diameter thereby forming an annular trough-like indentation in the area of the neck end of the housing. An electric vibrating device in the housing vibrates the head.

It is an objective of the present invention to provide a device for sexual stimulation that does not disclose its use by its appearance. It is a further objective to provide such a device that is easily transported and assembled for use. It is a still further objective of the invention to provide a sex toy that can be adapted to several different forms for varied types of stimulation. It is yet a further objective to provide such a device that can be made to operate on different voltage systems for international travel. Finally, it is an objective of the invention to provide a sexual stimulation device that is compact and inexpensive to manufacture.

While some of the objectives of the present invention are disclosed in the prior art, none of the inventions found include all of the requirements identified.

### SUMMARY OF THE INVENTION

The present invention addresses all of the deficiencies of vibrator sex toy conversion inventions and satisfies all of the objectives described above.

(1) A vibrator sex toy conversion, providing the desired features may be constructed from the following components. A vibrating device is provided. The vibrating device has a power source, a motion generator and a handle. An actuating member is provided. The actuating member is removably attached to the vibrating device.

(2) In a variant of the invention, the vibrating device is a handle portion of an electric toothbrush.

(3) In another variant, the actuating member has a proximal end and a distal end. The proximal end is adapted to fit the handle portion of the electric toothbrush. The distal end is sized and shaped for tactile stimulation. The actuating member replaces a removable toothbrush member that fits the handle portion.

(4) In still another variant, the distal end is formed of resilient material.

(5) In yet another variant, the distal end includes a plurality of ridges and grooves.

(6) In a further variant, the distal end includes a plurality resilient protruding elements.

(7) In yet a further variant, the actuating member is a cover adapted to fit removably over a portion of a removable toothbrush member fitting the handle portion.

(8) In still a further variant, the cover is formed of resilient material.

(9) In another variant of the invention, the cover includes a plurality of ridges and grooves.

(10) In a final variant of the invention, the cover comprises a plurality resilient protruding elements.

An appreciation of the other aims and objectives of the present invention and an understanding of it may be achieved by referring to the accompanying drawings and the detailed description of a preferred embodiment.

### DESCRIPTION OF THE DRAWINGS

FIG. 1 is a side elevational view of an electric toothbrush illustrating a cover over the brush element;

FIG. 2 is an exploded side elevational view of the FIG. 1 toothbrush illustrating a removable toothbrush member and the toothbrush handle;

FIG. 3 an exploded side elevational view of the preferred embodiment of the invention illustrating a detached actuating member having ridges and grooves;

FIG. 4 is a side elevational view of the FIG. 3 embodiment illustrating an actuating member having a plurality of resilient protruding elements;

FIG. 5 is a side elevational view of a second embodiment in which the actuating member is a replaceable cap for the toothbrush member having a plurality of resilient protruding elements;

FIG. 6 is a side elevational view of the FIG. 5 embodiment in which the actuating member is a replaceable cap for the toothbrush member having ridges and grooves;

FIG. 7 is an exploded side elevational view of the FIG. 3 embodiment illustrating an actuating member formed of resilient material;

FIG. 8 is an assembled side elevational view of the FIG. 7 embodiment.

### DETAILED DESCRIPTION OF THE PREFERRED EMBODIMENT

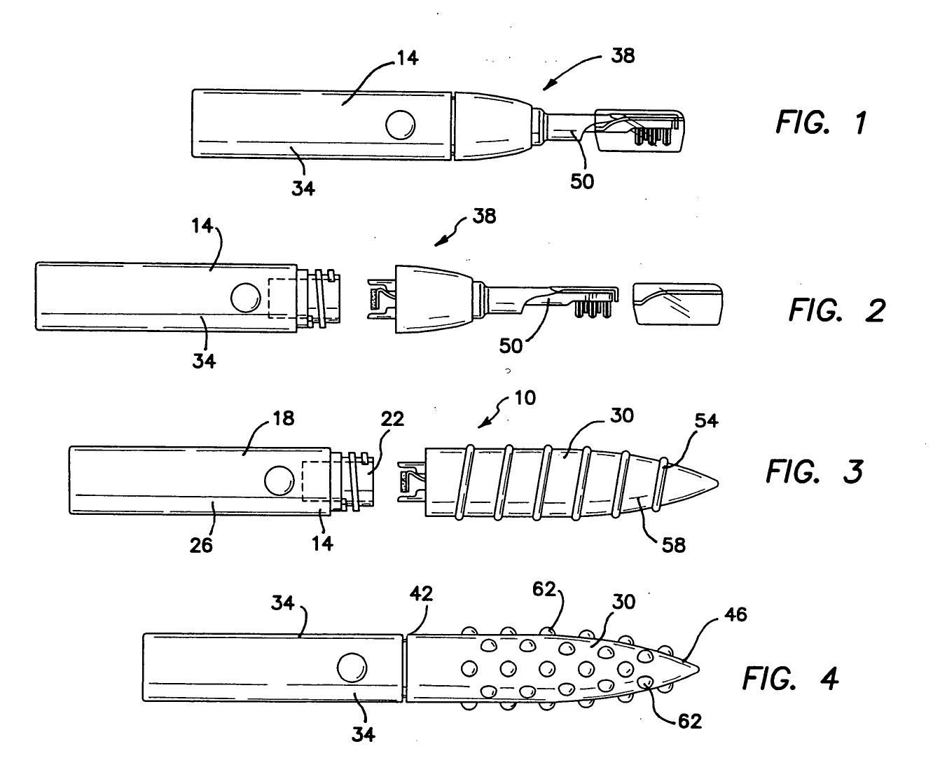

(1) FIGS. 1-8 illustrate a vibrator sex toy conversion 10, providing the desired features that may be constructed from the following components. As illustrated in FIG. 3, a vibrating device 14 is provided. The vibrating device 14 has a power source 18, a motion generator 22 and a handle 26. An actuating member 30 is provided. The actuating member 30 is removably attached to the vibrating device 14.

(2) In a variant of the invention, as illustrated in FIGS. 1 and 2, the vibrating device 14 is a handle portion 34 of an electric toothbrush 38.

(3) In another variant,.as illustrated in FIGS. 3, 4, 7 and 8, the actuating member 30 has a proximal end 42 and a distal end 46. The proximal end 42 is adapted to fit the handle portion 34 of the electric toothbrush 38. The distal end 46 is sized and shaped for tactile stimulation. The actuating member 30 replaces a removable toothbrush member 50 that fits the handle portion 34.

(4) In still another variant, the distal end 46 is formed of resilient material.

(5) In yet another variant, as illustrated in FIG. 3, the distal end 46 includes a plurality of ridges 54 and grooves 58.

(6) In a further variant, as illustrated in FIG. 4, the distal end 46 includes a plurality resilient protruding elements 62.

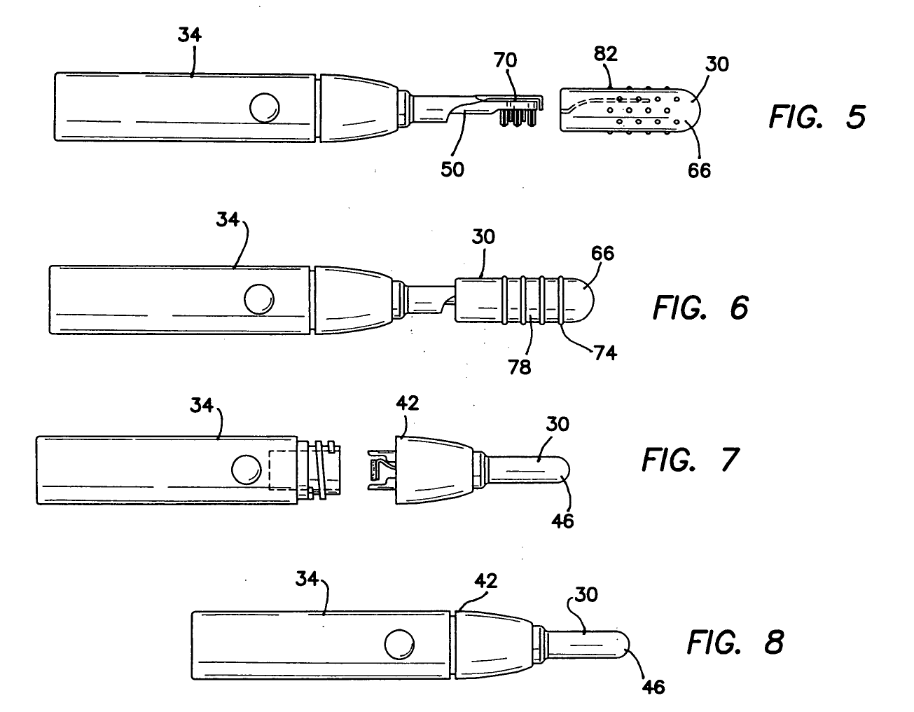

(7) In yet a further variant, as illustrated in FIGS. 5 and 6, the actuating member 30 is a cover 66 adapted to fit removably over a portion 70 of a removable toothbrush member 50 fitting the handle portion 34.

(8) In still a further variant, the cover 66 is formed of resilient material.

(9) In another variant of the invention, as illustrated in FIG. 6, the cover 66 includes a plurality of ridges 74 and grooves 78.

(10) In a final variant of the invention, as illustrated in FIG. 5, the cover 66 comprises a plurality resilient protruding elements 82.

The vibrator sex toy conversion 10 has been described with reference to particular embodiments. Other modifications and enhancements can be made without departing from the spirit and scope of the claims that follow.

1. A vibrator sex toy conversion, comprising:
a vibrating device, said vibrating device having a power source, a motion generator and a handle; said vibrating device being designed for use with a non-sexually stimulating actuating member; and
an sexually stimulating actuating member, said sexually stimulating actuating member being specifically designed for sexual stimulation and adapted for attachment to said vibrating device.

2. The vibrator sex toy conversion, as described in claim 1, wherein said vibrating device is a handle portion of an electric toothbrush.

3. The vibrator sex toy conversion, as described in claim 2, wherein said sexually stimulating actuating member has a proximal end and a distal end, said proximal end being adapted to fit said handle portion, and said distal end being sized and shaped for sexually stimulating tactile stimulation.

4. The vibrator sex toy conversion, as described in claim 3, wherein said distal end is formed of resilient material.

5. The vibrator sex toy conversion, as described in claim 3, wherein said distal end comprises a plurality of ridges and grooves.

6. The vibrator sex toy conversion, as described in claim 3, wherein said distal end comprises a plurality of resilient protrusions.

7. The vibrator sex toy conversion, as described in claim 2, wherein said sexually stimulating actuating member is a cover adapted to fit removably over the bristles and a portion of a removable toothbrush member fitting said handle portion.

8. The vibrator sex toy conversion, as described in claim 7, wherein said cover is formed of resilient material.

9. The vibrator sex toy conversion, as described in claim 7, wherein said cover comprises a plurality of ridges and grooves.

10. The vibrator sex toy conversion, as described in claim 7, wherein said cover comprises a plurality of resilient protrusions.

11. A sex toy, comprising: a sexually stimulating actuating member, said sexually stimulating actuating member being specifically designed for sexual stimulation and adapted for attachment to a vibrating device designed for use with a non-sexually stimulating actuating member; said vibrating device having a power source, a motion generator and a handle.

12. The sex toy, as described in claim 11, wherein said vibrating device is a handle portion of an electric toothbrush.

13. The sex toy, as described in claim 12, wherein said sexually stimulating actuating member has a proximal end and a distal end, said proximal end being adapted to fit said handle portion, and said distal end being sized and shaped for sexually stimulating tactile stimulation.

14. The sex toy, as described in claim 13, wherein said distal end is formed of resilient material.

15. The sex toy, as described in claim 13, wherein said distal end comprises a plurality of ridges and grooves.

16. The sex toy as described in claim 13, wherein said distal end comprises a plurality resilient protrusions.

17. The sex toy, as described in claim 12, wherein said sexually stimulating actuating member is a cover adapted to fit removably over the bristles and a portion of a removable toothbrush member fitting said handle portion.

18. The sex toy, as described in claim 17, wherein said cover is formed of resilient material.

19. The sex toy, as described in claim 17, wherein said cover comprises a plurality of ridges and grooves.

20. The sex toy, as described in claim 17, wherein said cover comprises a plurality of resilient protrusions.

21. A sex toy, comprising: a sexually stimulating actuating member, said sexually stimulating actuating member being specifically designed for sexual stimulation and adapted to fit removably over the bristles and a portion of the shaft of an electric toothbrush.

22. The sex toy, as described in claim 21, wherein said sexually stimulating actuating member is formed of resilient material.

23. The sex toy, as described in claim 21, wherein said sexually stimulating actuating member comprises a plurality of ridges and grooves.

24. The sex toy as described in claim 21, wherein said sexually stimulating actuating member comprises a plurality of resilient protrusions.
